(19) United States
(12) Patent Application Publication
(10) Pub. No.: US 2021/0154423 A1
(43) Pub. Date: May 27, 2021

(54) VENTILATION TRIGGER DETECTION METHOD AND APPARATUS, VENTILATION DEVICE

(71) Applicants: SHENZHEN MINDRAY BIO-MEDICAL ELECTRONICS CO., LTD., Shenzhen (CN); Peking Union Medical College Hospital, Chinese Academy of Medic, Beijing (CN)

(72) Inventors: Jun XU, Beijing (CN); Jinglei LIU, Shenzhen (CN); Xuezhong YU, Beijing (CN); Yangyang FU, Beijing (CN); Xinru ZOU, Shenzhen (CN); Xiaoyong ZHOU, Shenzhen (CN)

(73) Assignees: SHENZHEN MINDRAY BIO-MEDICAL ELECTRONICS CO., LTD., Shenzhen (CN); Peking Union Medical College Hospital, Chinese Academy of Medical Science and Peking Union Medical C, Beijing (CN)

(21) Appl. No.: 17/169,524

(22) Filed: Feb. 7, 2021

Related U.S. Application Data
(63) Continuation of application No. PCT/CN2018/101606, filed on Aug. 21, 2018.

(51) Int. Cl. A61M 16/00

(52) U.S. Cl. CPC A61M 16/024; A61M 2205/502; A61M 2230/40; A61M 2016/0027; A61M 2016/0033; A61M 2205/52

(57) ABSTRACT

A ventilation trigger detection method performed by a ventilation device is disclosed. The method includes monitoring a ventilation parameter during mechanical ventilation for a user, the ventilation parameter including at least one of an airway pressure and an airway flow, and determining patient-ventilator synchrony during the ventilation according to a change in the ventilation parameter. Furthermore, this disclosure also relates to a ventilation trigger detection apparatus, a ventilation device, and a storage medium.

### CROSS-REFERENCE TO RELATED APPLICATIONS

This application is a continuation of PCT Application NO. PCT/CN2018/101606, filed Aug. 21, 2018, entitled “VENTILATION TRIGGER DETECTION METHOD AND APPARATUS, VENTILATION DEVICE, AND STORAGE MEDIUM,” the entire contents of which are incorporated herein by reference.

### TECHNICAL FIELD

The disclosure relates to the field of medical technologies, and in particular to a ventilation trigger detection method and apparatus, a ventilation device and a storage medium.

### BACKGROUND

Ventilators, as an effective means capable of artificially replacing a function of spontaneous ventilation, have been widely used in respiratory failure, anesthesia and respiration management, respiratory support therapy and emergency resuscitation caused by various causes. During mechanical ventilation for users by using the ventilators, patient-ventilator incoordination or patient-ventilator out-of-synchronization caused by abnormal ventilation (such as ineffective effort) often occurs, and the ventilators in the related art may not identify abnormal events or patient-ventilator out-of-synchronization during the ventilation.

### SUMMARY

In view of this, this disclosure provides a ventilation trigger detection method and apparatus, a ventilation device, which are capable of accurately determining the patient-ventilator synchrony during mechanical ventilation.

In order to achieve the above objects, technical solutions of the embodiments of the disclosure are implemented as follows:

In a first aspect, an embodiment of this disclosure provides a ventilation trigger detection method, may be applied to a ventilation device. The method may include:

monitoring a ventilation parameter during mechanical ventilation for a user, the ventilation parameter may include at least one of an airway pressure and an airway flow; and determining patient-ventilator synchrony during the ventilation according to a change in the ventilation parameter.

In the solution described above, the step of determining the patient-ventilator synchrony during the ventilation according to the change in the ventilation parameter may include:

analyzing a change trend of the obtained ventilation parameter; and

determining whether patient-ventilator out-of-synchronization occurs during the ventilation according to the change trend of the ventilation parameter.

In the solution described above, after it is determined that the patient-ventilator out-of-synchronization occurs during the ventilation according to the change trend of the ventilation parameter, the method may further include:

determining the type of the patient-ventilator out-of-synchronization according to the change trend of the ventilation parameter.

In the solution described above, the type of the patient-ventilator out-of-synchronization may include one or more of ineffective effort, double trigger, delayed cycling and premature cycling.

In the solution described above, the step of determining whether the ineffective effort occurs according to the change trend of the ventilation parameter may include:

determining the occurrence of the ineffective effort if a valley appears in an airway pressure-time waveform and/or an accelerated rise appears in a ventilation flow-time waveform at an expiratory stage, and inspiratory trigger of a ventilator is not enabled.

In the solution described above, the step of determining whether the double trigger occurs according to the change trend of the ventilation parameter may include:

determining the occurrence of the double trigger if two inspiratory pressure waveforms appear in the airway pressure-time waveform at an inspiratory stage and/or a short-time expiratory cycle appears between two inspiratory cycles in the airway flow-time waveform.

In the solution described above, the step of determining whether the delayed expiratory occurs according to the change trend of the ventilation parameter may include:

determining the occurrence of the delayed expiratory if a rise appears in the airway pressure-time waveform or an accelerated drop occurs in a ventilation flow-time waveform at an inspiration-to-expiration transitional stage.

In the solution described above, the step of determining whether the early expiratory occurs according to the change trend of the ventilation parameter may include:

determining the occurrence of the early expiratory if a non-monotonic drop appears in the airway pressure-time waveform or a non-monotonic rise appears in the airway flow-time waveform at the inspiration-to-expiration transitional stage.

In the solution described above, after the type of the patient-ventilator out-of-synchronization is determined according to the change trend of the ventilation parameter, the method further may include:

adjusting ventilation trigger setting of the ventilation device or outputting prompt information about the patient-ventilator out-of-synchronization according to the determined type of the patient-ventilator out-of-synchronization.

In the solution described above, the inspiratory trigger sensitivity of the ventilation device may be reduced when the ineffective effort occurs, or the inspiratory trigger may be enabled when it is detected that the valley appears in the airway pressure-time waveform and/or the accelerated rise appears in the ventilation flow-time waveform;

inspiratory time, an inspiratory pressure or a tidal volume may be increased when the double trigger occurs;

the inspiratory trigger sensitivity of the ventilation device may be increased when the delayed expiratory occurs; and

the inspiratory trigger sensitivity of the ventilation device may be reduced when the early expiratory occurs.

In a second aspect, an embodiment of the disclosure further provides a ventilation trigger detection apparatus, applied to a ventilation device. The apparatus may include:

a parameter monitoring unit may be configured to monitor a ventilation parameter during mechanical ventilation for a user, the ventilation parameter may include at least one of an airway pressure and an airway flow; and

a processing unit may be configured to determine patient-ventilator synchrony during the ventilation according to a change in the ventilation parameter.

In the solution described above, the processing unit may be further configured to analyze a change trend of the obtained ventilation parameter;

and to determine whether patient-ventilator out-of-synchronization occurs during the ventilation according to the change trend of the ventilation parameter.

In the solution described above, the processing unit may be further configured to determine the type of the patient-ventilator out-of-synchronization according to the change trend of the ventilation parameter.

In the solution described above, the type of the patient-ventilator out-of-synchronization may include one or more of ineffective effort, double trigger, false inspiratory trigger, delayed cycling and premature cycling.

In the solution described above, the processing unit may be further configured to determine the occurrence of the ineffective effort when detecting that a valley appears in an airway pressure-time waveform and/or an accelerated rise appears in a ventilation flow-time waveform at an expiratory stage, and inspiratory trigger of a ventilator is not enabled.

In the solution described above, the processing unit may be further configured to determine the occurrence of the double trigger when detecting that two inspiratory pressure waveforms appear in the airway pressure-time waveform at an inspiratory stage and/or a short-time expiratory cycle appears between two inspiratory cycles in the airway flow-time waveform.

In the solution described above, the processing unit may be further configured to determine the occurrence of the delayed expiratory when detecting that a rise appears in the airway pressure-time waveform or an accelerated drop occurs in a ventilation flow-time waveform at an inspiration-to-expiration transitional stage.

In the solution described above, the processing unit may be further configured to determine the occurrence of the early expiratory when detecting that a non-monotonic drop appears in the airway pressure-time waveform or a non-monotonic rise appears in the airway flow-time waveform at the inspiration-to-expiration transitional stage.

In the solution described above, the processing unit may be further configured to adjust ventilation trigger setting of the ventilation device or output prompt information about the patient-ventilator out-of-synchronization according to the determined type of the patient-ventilator out-of-synchronization.

In a third aspect, an embodiment of the disclosure further provides a ventilation device, may include a ventilation trigger detection apparatus provided by the embodiment of the disclosure, a gas source, an inspiratory branch, an expiratory branch, a respiration line and a controller, where

the gas source may supply gas during mechanical ventilation;

the inspiratory branch may be connected to the gas source to provide an inspiration path during the mechanical ventilation;

the expiratory branch may provide an expiration path during the mechanical ventilation;

the respiration line may be connected to the inspiratory branch and the expiratory branch respectively, and used for delivering gas to a user or exhausting gas from a user during the mechanical ventilation; and

the ventilation trigger detection apparatus may be connected to the inspiratory branch, the expiratory branch and the controller respectively.

In a fourth aspect, an embodiment of the disclosure may further provide a ventilation trigger detection apparatus. The ventilation trigger detection apparatus may include:

a memory may be configured to store executable instructions; and

s a processor may be configured to implement a ventilation trigger detection method provided by the embodiment of the disclosure when executing the executable instructions stored in the memory.

In a fifth aspect, an embodiment of the disclosure may further provide a storage medium storing executable instructions. The executable instructions may be configured to implement a ventilation trigger detection method provided by the embodiment of the disclosure when being executed by a processor.

By applying the ventilation trigger detection method and apparatus, the ventilation device and the storage medium of the embodiments of the disclosure, during the mechanical ventilation for the user, the patient-ventilator synchrony during the ventilation is determined by monitoring the ventilation parameter to obtain the change in the ventilation parameter over time. In this way, the user may find, in a timely manner, the patient-ventilator out-of-synchronization of the ventilator during the mechanical ventilation, and then make corresponding adjustments in a timely manner to better realize the patient-ventilator synchronization.

### BRIEF DESCRIPTION OF THE DRAWINGS

FIG. 1A is a first schematic diagram of a composition structure of a ventilation trigger detection apparatus provided by an embodiment of the disclosure;

FIG. 1B is a schematic diagram of a composition structure of a ventilation device provided by an embodiment of the disclosure;

FIG. 2 is a schematic diagram of a waveform of one respiratory cycle of a ventilator in a pressure trigger mode provided by an embodiment of the disclosure;

FIG. 3 is a schematic flowchart of a ventilation trigger detection method provided by an embodiment of the disclosure;

FIG. 4 is a schematic diagram of waveforms of pressure trigger and flow trigger in a pressure support mode provided by an embodiment of the disclosure;

FIG. 5 is a schematic diagram of waveforms of normal inspiration-to-expiration switching in the pressure support mode provided by an embodiment of the disclosure;

FIG. 6 is a schematic diagram of a flow-time waveform of ineffective effort in the pressure support mode;

FIG. 7 is a schematic diagram of waveforms of ineffective effort in the pressure support mode provided by an embodiment of the disclosure;

FIG. 8 is a schematic diagram of waveforms of double trigger provided by an embodiment of the disclosure;

FIG. 9 is a schematic diagram of waveforms of false trigger provided by an embodiment of the disclosure;

FIG. 10 is a schematic diagram of waveforms of delayed cycling provided by an embodiment of the disclosure;

FIG. 11 is a schematic diagram of waveforms of premature cycling provided by an embodiment of the disclosure; and

FIG. 12 is a second schematic diagram of a composition structure of the ventilation trigger detection apparatus provided by the embodiment of the disclosure.

### DESCRIPTION OF THE PREFERRED EMBODIMENTS

The disclosure will be further described below in detail in conjunction with the accompanying drawings and the embodiments. It should be understood that the embodiments provided herein are merely intended to explain the disclosure, and are not intended to limit the disclosure. In addition, the embodiments provided below are used to implement some embodiments of the disclosure, but not all embodiments for implementing the disclosure. In the case of no conflict, the technical solutions recorded in the embodiments of the disclosure may be implemented in any combination.

It should be noted that, in the embodiments of the disclosure, the terms “comprise”, “include” or any other variation thereof are intended to cover non-exclusive inclusion, so that a method or apparatus including a series of elements includes not only explicitly recorded elements, but also other elements not explicitly listed, or elements inherent in implementing the method or apparatus. In the absence of more restrictions, the element defined by the phrase “including a/an . . . ” does not exclude the presence of a further related element (for example, steps in the method or units in the apparatus, wherein the unit may be a partial circuit, a partial processor, a partial program, software, or the like) in the method or apparatus that includes the element.

It should be noted that the term “first/second/third” in the embodiments of the disclosure is only used to distinguish similar objects, and does not represent specific order for the objects. It may be understood that “first/second/third” may be interchanged for specific order or chronological order when allowed. It should be understood that the objects distinguished by “first/second/third” may be interchangeable where appropriate, so that the embodiments of the disclosure described herein can be implemented in an order other than that illustrated or described herein.

It has been found that during mechanical ventilation, a patient needs, when spontaneously respiring, to make an inspiratory or expiratory effort to reach a set inspiratory trigger (which may be set by setting a pressure or flow trigger sensitivity)/expiratory switching (which may be set according to a percentage of an inspiratory flow peak) sensitivity so that a ventilator can be switched to a corresponding inspiratory or expiratory phase. For example, the inspiratory trigger may be set as flow trigger, the inspiratory phase may be enabled when a flow exceeds a trigger sensitivity (e.g., 2 L/min), or in a pressure trigger mode, the inspiratory phase may be enabled when an airway pressure is below a positive end expiratory pressure (PEEP)-trigger sensitivity (e.g., 2 cmH2O). The expiratory trigger sensitivity may be generally a percentage of an inspiratory peak flow, for example, the ventilator may be switched to the expiratory phase when the inspiratory flow decreases to 25% of the inspiratory peak flow. Since the inspiratory or expiratory trigger sensitivity is set by a doctor based on experience, the situation clinically may occur that the trigger sensitivity setting of the ventilator may be inconsistent with the demand of a patient, resulting in the occurrence of patient-ventilator out-of-synchronization events, such as ineffective effort, double trigger, delayed cycling, premature cycling and delayed inspiratory, and then the use effect of the user may be influenced.

Before describing the embodiments of the disclosure in further detail, the nouns and terms involved in the embodiments of the disclosure are explained, and the nouns and terms involved in the embodiments of the disclosure are applicable to the following explanation.

(1) Flow trigger may refer to that a continuous air flow is delivered in a ventilator loop, and a ventilator detects air flow velocities at an inlet end and an outlet end of a breathing circuit, and is triggered to deliver gas when a difference between the air flow velocities at the two ends reaches a preset level.

(2) Pressure trigger may refer to that a pressure in an airway drops when a user inhales, the ventilator detects the pressure change and starts gas delivery, so that synchronous inhalation is completed.

(3) Tidal volume may refer to the volume of gas inhaled or exhaled each time when the user breathes quietly. It is related to the age, gender, volume surface, breathing habits and body metabolism of the user. The tidal volume set by the ventilator is usually referred to as an inspired gas volume and may be adjusted according to the blood gas analysis of the user.

(4) Patient-ventilator out-of-synchronization may refer to that a respiratory cycle of a ventilation device, such as a ventilator, is not coordinated with a patient.

A ventilation trigger detection apparatus provided by an embodiment of the disclosure will be described below. The ventilation trigger detection apparatus provided by the embodiment of the disclosure may be implemented in hardware, software or a combination of hardware and software, and various exemplary implementations of the ventilation trigger detection apparatus provided in the embodiment of the disclosure will be described below.

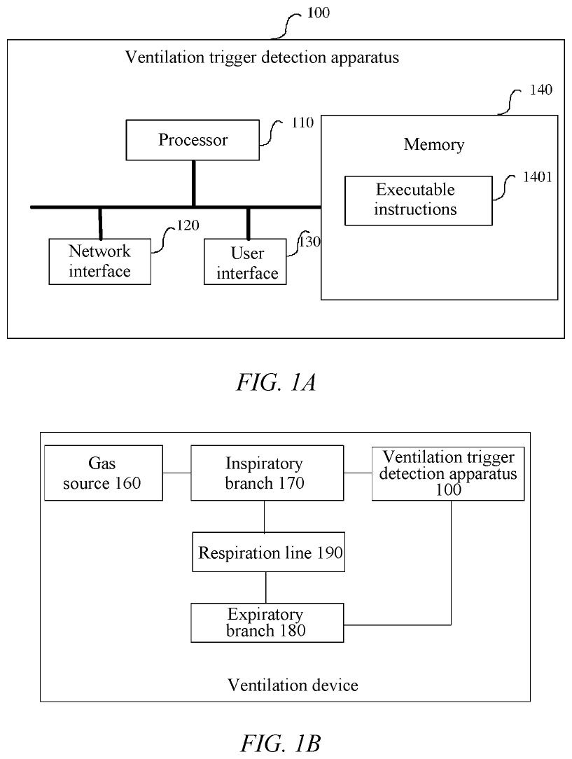

A hardware structure of the ventilation trigger detection apparatus of the embodiment of the disclosure will be described in detail below. FIG. 1A is a schematic diagram of a composition structure of the ventilation trigger detection apparatus provided by the embodiment of the disclosure, and it will be understood that FIG. 1A only shows an exemplary structure, rather than all the structures, of the ventilation trigger detection apparatus, and that part or all of the structure shown in FIG. 1A may be implemented according to requirements. The ventilation trigger detection apparatus 100 provided by the embodiment of the disclosure may include: at least one processor 110, a memory 140, and a user interface 130. The ventilation trigger detection apparatus 100 may be further provided with a network interface 120 according to actual requirements.

The user interface 130 may include a display, a keyboard, a mouse, a trackball, a click wheel, keys, buttons, a touch pad, or a touch screen, etc.

The memory 140 may be a volatile memory or a non-volatile memory, or may include both a volatile memory and a non-volatile memory. The nonvolatile memory may be a read only memory (ROM), a programmable read-only memory (PROM), an erasable programmable read-only memory (EPROM), a flash memory, etc. The volatile memory may be a random access memory (RAM), which acts as an external cache. By way of example, and not limitation, many forms of RAMs are available, such as a static random access memory (SRAM), and a synchronous static random access memory (SSRAM). The memory 140 described in the embodiment of the disclosure is intended to include these memories and any other suitable types of memories.

The processor 110 may be an integrated circuit chip having a signal processing capability, such as a general-purpose processor, a digital signal processor (DSP), or other programmable logic devices, discrete gates or transistor logic devices and discrete hardware components, wherein the general-purpose processor may be a microprocessor or any conventional processor.

The memory 140 is capable of storing executable instructions 1401 to support operations of the ventilation trigger detection apparatus 100. Examples of these executable instructions may include: various forms of software modules, such as programs, plug-ins and scripts, configured to operate on the ventilation trigger detection apparatus 100. The programs, for example, may include an operating system and application programs, wherein the operating system contains various system programs, such as a framework layer, a core library layer and a driver layer, which are configured to implement various basic services and to process hardware-based tasks.

As an example, implemented by combining software and hardware, of the ventilation trigger detection apparatus provided by the embodiment of the disclosure, the ventilation trigger detection apparatus provided by the embodiment of the disclosure may be directly embodied as different forms of software modules executed by the processor 110, the software modules may be located in a storage medium, the storage medium may be located in the memory 140, and the processor 110 reads executable instructions included in the software modules in the memory 140 and implements a ventilation trigger detection method provided by the embodiments of the disclosure in combination with necessary hardware (for example, including the processor 110 and other components connected to a bus).

A ventilation device provided by the embodiment of the disclosure will be described below. FIG. 1B is a schematic diagram of a composition structure of the ventilation device provided by the embodiment of the disclosure. Referring to FIG. 1B, the ventilation device provided by the embodiment of the disclosure includes a ventilation trigger detection apparatus 100 provided by the embodiment of the disclosure, and a gas source 160, an inspiratory branch 170, an expiratory branch 180 and a respiration line 190; wherein the gas source may supply gas during mechanical ventilation; the inspiratory branch may be connected to the gas source to provide an inspiration path during the mechanical ventilation; the expiratory branch may provide an expiration path during the mechanical ventilation; the respiration line may be connected to the inspiratory branch and the expiratory branch respectively, and used for delivering gas to a user or exhausting gas from a user during the mechanical ventilation; and the ventilation trigger detection apparatus may be connected to the inspiratory branch and the expiratory branch respectively. The ventilation device provided by the embodiment of the disclosure may be a ventilator, an anesthesia machine or other devices.

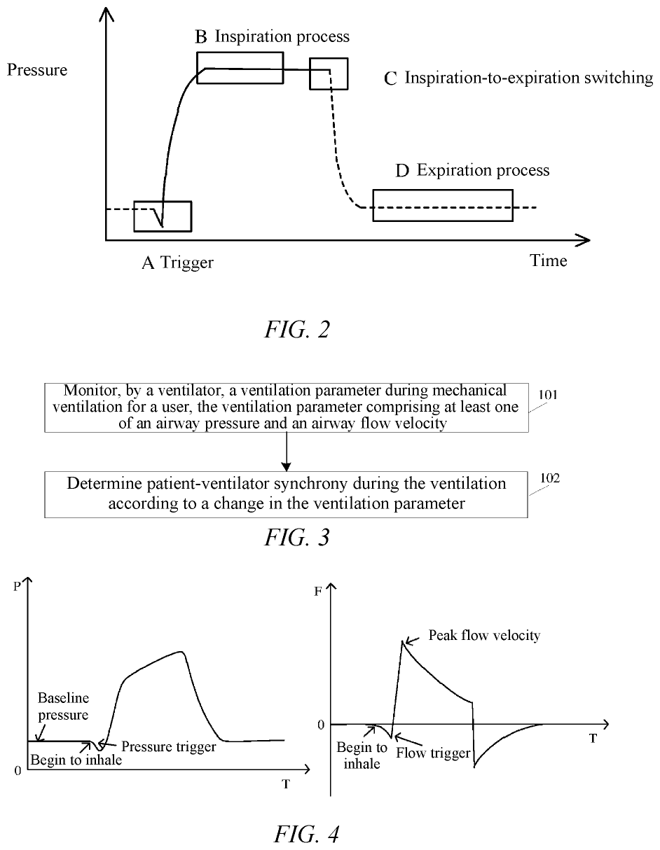

In the following, the mechanical ventilation is illustrated by taking a ventilator as an example of the ventilation device. FIG. 2 is a schematic diagram of a waveform of one respiratory cycle of the ventilator in a pressure trigger mode provided by the embodiment of the disclosure. Referring to FIG. 2, one respiratory cycle of mechanical ventilation of the ventilator may include inspiratory trigger (starting the gas delivery of the ventilator), inspiration process (gas delivery process of the ventilator), inspiration-to-expiration switching (ending the gas delivery of the ventilator) and expiration process.

In this case, a patient actively inhales, causing a pressure drop or flow change in an airway, and the ventilator may sense the inhalation action of the user and give the user one delivery of gas, which is called a user trigger. The sensing action of the ventilator may be set manually and may be controlled by adjusting the trigger sensitivity. Trigger modes of the ventilator may include, but are not limited to, flow trigger and pressure trigger.

In the inspiration process, the ventilator may output gas at a certain flow, and a certain volume and a certain pressure are generated as the gas enters a breathing circuit and the user's lungs. The inspiration-to-expiration switching of the ventilator may be controlled in the following three modes:

controlling ventilation (volume/pressure), namely enabling the ventilator to provide a constant ventilation tidal volume or pressure to the patient for ventilation, and performing time-based switching, namely performing switching when the gas delivery time reaches a set inspiratory time; and

pressure support, namely the user obtains a certain level of pressure support after triggering the ventilator to deliver the gas, and performing flow-based switching, namely performing switching when the flow drops to a percentage of a peak flow.

Continuing to take the ventilator as the example of the ventilation device, the ventilation trigger detection method provided by the embodiment of the disclosure will be described. Referring to FIG. 3, FIG. 3 is an optional flowchart of the ventilation trigger detection method provided by the embodiment of the disclosure. The ventilation trigger detection method of the embodiment of the disclosure will be described with reference to the steps shown in FIG. 3.

Step 101, monitoring, by a ventilator, a ventilation parameter during mechanical ventilation for a user, the ventilation parameter may include at least one of an airway pressure and an airway flow; and

Step 102, determining patient-ventilator synchrony during the ventilation according to a change in the ventilation parameter.

In an embodiment, the ventilator may monitor an airway pressure and an airway flow simultaneously so as to learn changes in the airway pressure and the airway flow over time, for example, the ventilator may acquire waveforms of the changes in the airway pressure and the airway flow over time and learns the changes in the airway pressure and the airway flow over time by analyzing the waveforms.

During actual implementation, the ventilator may detect a change in the ventilation parameter, and determine that ventilation trigger is abnormal, that is, patient-ventilator out-of-synchronization, when the change in the ventilation parameter meets a corresponding parameter change condition. Specifically, it may be determined in the following way that the change in the ventilation parameter meets the corresponding parameter change condition: acquiring a waveform of a ventilation parameter over time; carrying out similarity matching on the acquired waveform and a stored waveform of the corresponding parameter; and determining that the change in the ventilation parameter meets the corresponding parameter change condition when the similarity obtained by the matching reaches a waveform similarity threshold.

In another embodiment, the ventilator may also determine in the following way that the change in the ventilation parameter meets the corresponding parameter change condition: acquiring a waveform of a ventilation parameter over time; analyzing a change trend of the acquired waveform; and determining that the change in the ventilation parameter meets the corresponding parameter change condition when the change trend of the waveform is the same as a stored change trend of a waveform of the corresponding parameter.

During actual implementation, abnormal ventilation of the ventilator during the mechanical ventilation may include, but is not limited to an abnormal trigger event and an abnormal inspiration-to-expiration switching event. The ventilator may detect the abnormal trigger event and the abnormal inspiration-to-expiration switching event, wherein the types of abnormal trigger may include, but are not limited to: ineffective effort, double trigger and false trigger, and the types of abnormal inspiration-to-expiration switching may include, but are not limited to: delayed cycling and premature cycling. After determining the type of abnormal ventilation that has occurred on the ventilator, the ventilator may adjust the trigger sensitivity of the ventilator correspondingly according to the determined type of abnormal ventilation.

Before the patient-ventilator out-of-synchronization during the mechanical ventilation of the ventilator is described, normal trigger of gas delivery and normal inspiration-to-expiration switching of the ventilator are first described.

FIG. 4 is a schematic diagram of waveforms of pressure trigger and flow trigger in a pressure support mode provided by an embodiment of the disclosure. Referring to FIG. 4, the airway pressure may be reduced when the user inhales, and the ventilator may be triggered to enable an inspiratory phase and to start gas delivery when the airway pressure reaches a pressure trigger sensitivity; and the air flow velocities at the inlet end and the outlet end in the breathing circuit have an air flow difference when the user inhales, and the ventilator may be triggered to enable the inspiratory phase and to start gas delivery when the flow difference reaches a preset trigger sensitivity.

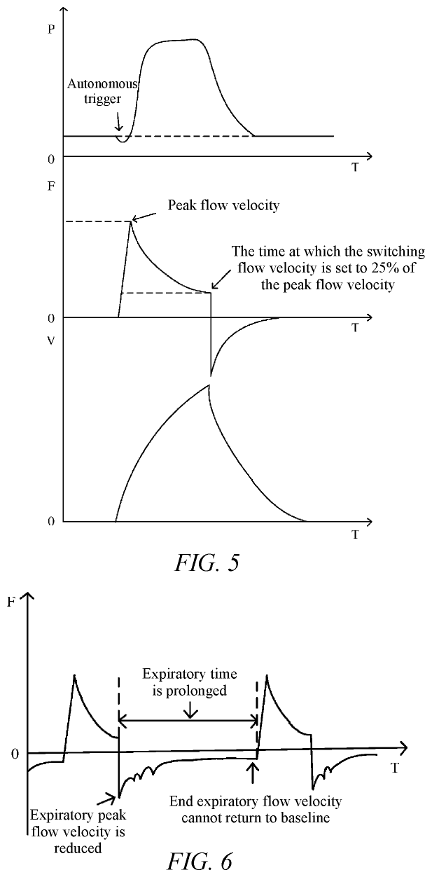

In volume control and pressure control modes, time-based switching may be performed by setting inspiratory time or an inspiration/expiration ratio, a respiratory rate, etc. Under the pressure support, a flow-based switching mode may be adopted, for example, reducing the flow to 25% of a peak flow serves as an index of flow-based switching. FIG. 5 is a schematic diagram of waveforms of normal inspiration-to-expiration switching in the pressure support mode provided by an embodiment of the disclosure. Referring to FIG. 5, the ventilator may perform inspiration-to-expiration switching when detecting that the flow reaches 25% of the peak flow.

In the following, the abnormal trigger event and the abnormal inspiration-to-expiration switching event in the patient-ventilator out-of-synchronization are explained respectively.

With regard to ineffective effort, the situation that the user has made an inhalation effort but cannot trigger the ventilator to effectively delivery gas is called ineffective effort. The ineffective effort may lead to patient-ventilator incoordination, so that inhalation work is increased, but the ventilator cannot be effectively triggered to deliver the gas, that is, trigger failure. Reasons for the ineffective efforts may include, but are not limited to, the following situations:

(1) decreased respiratory center drive: it may occur in sedation, hyperventilation, deep sleep and so on, the respiratory center drive of such populations decreases, inspiratory actions slow down, the trigger time is prolonged, and the occurrence rate of the ineffective effort increases;

(2) respiratory muscle weakness: in some disease states, the user suffers from respiratory muscle weakness, so that the inspiratory volume is insufficient to cause a pressure change in the line or cause the change in the flow to reach a trigger point, leading to ineffective effort, for example, it occurs when myasthenia gravis, Guillain-Barre syndrome and so on affect the respiratory muscle;

(3) too high trigger setting: when the trigger setting is too high, the work required to reach the trigger point increases, often leading to trigger difficulty.

(4) PEEPi: when the PEEPi occurs in the user, an end expiratory alveolar pressure increases, and the patient needs to strive to inhale to enable the alveolar pressure to reach a zero point and then drop to the trigger point such that the ventilator can be triggered to deliver the gas, increasing the work of the respiratory muscle and making trigger difficult. It is common in patients suffering from chronic obstructive pulmonary disease (COPD) and tachypnea, mostly caused by prolonged expiratory time and insufficient expiratory time due to increased expiratory resistance. The waveform is characterized by returning to a baseline by a flow-time curve method. Reference is made to FIG. 6, which is a schematic diagram of a flow-time waveform of the ineffective effort occurring in a patient with PEEPi in the pressure support mode.

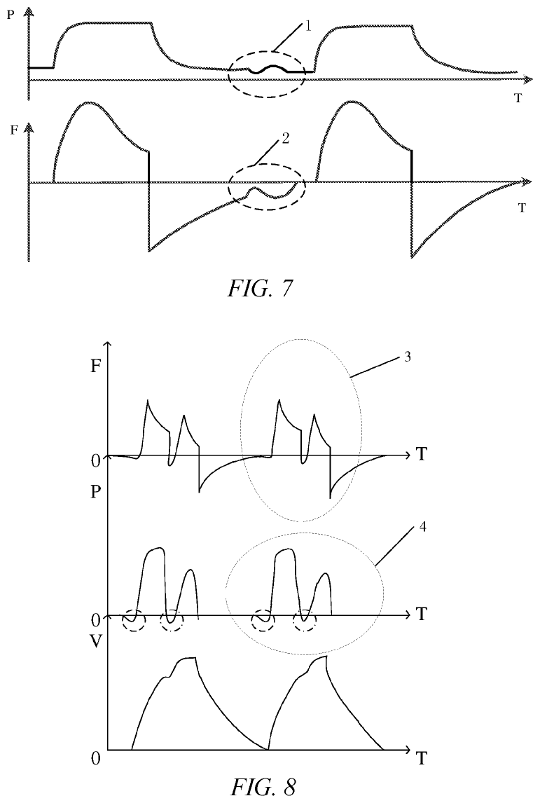

In the case of the ineffective effort described above, there are similar manifestations in the waveform of the airway pressure over time and the waveform of the airway flow over time. FIG. 7 is a schematic diagram of waveforms of ineffective effort in the pressure support mode provided by an embodiment of the disclosure. Referring to FIG. 7, parts indicated by reference numbers 1 and 2 show waveforms of the ineffective effort, which represent that the user performs an inhalation operation, but since the trigger sensitivity is not reached, the part indicated by the reference number 1 shows the situation that the pressure drops and then rises, the part indicated by the reference number 2 shows the situation that the slope of the flow suddenly increases (exceeds a preset threshold but does not reach the trigger sensitivity) and then becomes smaller, and the time corresponding to a turning point of the pressure change indicated by the reference number 1 and the moment of sudden change in the slope of the flow indicated by the reference number 2 may be the corresponding time of occurrence of an ineffective effort event.

In an embodiment, when the ventilator detects that at least one of the following situations has occurred by analyzing waveforms of the acquired ventilation parameters (the airway pressure and the airway flow) over time, it may be determined that an abnormal event of ineffective effort has occurred on the ventilator, that is, it may be determined that the change in the ventilation parameters meets corresponding parameter change conditions:

the change trend of the airway pressure over time at the expiratory stage may be drop and then rise, and a minimum value of the airway pressure in a first time period may be greater than the PEEP-pressure trigger sensitivity of the ventilator; or

the change rate of the airway flow over time at the expiratory stage appears at a time point when the change rate exceeds a change rate threshold, and the airway flow corresponding to the time point is smaller than the flow trigger sensitivity of the ventilator.

Accordingly, in the above situations, the time corresponding to the turning point at which the pressure at the expiratory stage drops and then rises, or the time at which the change rate of the airway flow over time at the expiratory stage exceeds the change rate threshold may be the time of occurrence of an ineffective effort.

In an embodiment, the ventilator may determine the occurrence of the ineffective effort when detecting that a valley appears in an airway pressure-time waveform and/or an accelerated rise appears in a ventilation flow-time waveform at an expiratory stage, and inspiratory trigger of the ventilator is not enabled.

In an embodiment, a pressure-time waveform graph and a flow-time waveform graph corresponding to the ineffective effort event may be stored in the ventilator, a corresponding waveform graph may be obtained by monitoring the airway pressure and/or airway flow during the mechanical ventilation by the ventilator, and the obtained waveform graph may be matched with the stored corresponding waveform graph of ineffective effort. For example, the obtained pressure-time waveform graph may be matched with the stored pressure-time waveform graph corresponding to the ineffective effort, and when the similarity obtained by matching reaches a similarity threshold (e.g., 0.9), it may be determined that an abnormal event of ineffective effort has occurred on the ventilator, and thus abnormality prompt information indicating that ineffective effort has occurred on the ventilator may be sent.

In an embodiment, the ventilator may be triggered to enable the inspiratory phase, that is, the ventilator may be triggered to start gas delivery when determining that the abnormal event of ineffective effort has occurred on the ventilator.

In an embodiment, the ventilator may send abnormality prompt information, by means of a user interface (UI) and/or in the form of sound, indicating that the ineffective effort has occurred on the ventilator when determining that the abnormal event of ineffective effort has occurred, so that the user may adjust the trigger sensitivity to better realize the patient-ventilator synchronization.

In an embodiment, a preset trigger sensitivity adjusting strategy may be adopted to adjust the trigger sensitivity of the ventilator when the ventilator determines that the abnormal event of ineffective effort has occurred, for example, the trigger sensitivity may be periodically reduced (e.g., in each subsequent respiratory cycle, the trigger sensitivity is reduced to 90% of that in a previous cycle each time) until no ineffective effort event occurs. Alternatively, trigger determination may be carried out by determining the change trend of the flow or pressure waveform, if the slope of the flow gradually increases to a certain threshold, it may be considered that an inhalation effort has made to trigger the ventilator to delivery gas.

With regard to double trigger, due to improper setting of the trigger sensitivity of the ventilator, the ventilator may be repeatedly triggered within a short time (e.g., 1 s), causing patient-ventilator incoordination, the patient has breathing difficulty and cannot reach a preset tidal volume or minute ventilation volume of the ventilator, and the ventilation quality may be reduced. Reasons for the double trigger may include, but are not limited to, the following situations:

(1) too low inspiratory flow: the inhaling action will be made again when the flow of the ventilator is set improper to make the patient feel that the delivery flow cannot meet the demand of the body, so that the inspiratory trigger sensitivity is achieved, and the ventilator is triggered again to delivery gas;

(2) too low tidal volume: the patient will have to inhale again when the tidal volume of the ventilator is set too low to meet the demand of the patient, and after the trigger sensitivity is reached, the ventilator is triggered to delivery gas;

(3) improper setting of expiration switching: the inhalation effort is again initiated, resulting in repeated trigger of the ventilator, when the ventilator performs premature switching to cause the patient to inhale not enough gas to meet his/her own need.

In the case of the double trigger described above, there are similar manifestations in the waveform of the airway pressure over time and the waveform of the airway flow over time. FIG. 8 is a schematic diagram of waveforms of double trigger provided by an embodiment of the disclosure. Referring to FIG. 8, the ventilator may be triggered twice to enable the inspiratory phase in one respiratory cycle, as shown by parts indicated by the reference numbers 3-4 in FIG. 8.

In an embodiment, when the ventilator detects that at least one of the following situations has occurred by analyzing waveforms of the acquired ventilation parameters (the airway pressure and the airway flow) over time, it may be determined that an abnormal event of double trigger has occurred on the ventilator, that is, it may be determined that the change in the ventilation parameters meets corresponding parameter change conditions:

the airway pressure may reach the pressure trigger sensitivity at least twice in a third time period; and

the airway flow may reach the flow trigger sensitivity at least twice in the third time period.

In practical application, the third time period described herein may be set according to actual requirements, for example, the third time period may be set to correspond to an exhalation time constant of the patient.

In an embodiment, the ventilator may determine the occurrence of the double trigger when detecting that two sections of inspiratory pressure waveforms appear in the airway pressure-time waveform at an inspiratory stage and/or a short-time expiratory cycle appears between two inspiratory cycles in the airway flow-time waveform. The user's expiratory stage herein may be defined by a waveform, and the part in the waveform other than the expiratory stage is the inspiratory stage; and the term “short-time” refers to a time smaller than a preset time threshold, and the time threshold value may be set according to actual demands, such as twice the time constant, wherein the time constant is equal to the product of an airway resistance and a compliance.

In an embodiment, a pressure-time waveform graph and a flow-time waveform graph corresponding to the double trigger event may be stored in the ventilator, a corresponding waveform graph may be obtained by monitoring the airway pressure and/or airway flow during the mechanical ventilation by the ventilator, and the obtained waveform graph may be matched with the stored corresponding waveform graph of double trigger. For example, the obtained pressure-time waveform graph may be matched with the stored pressure-time waveform graph (within one respiratory cycle) corresponding to the double trigger, and when the similarity obtained by matching reaches a similarity threshold (e.g., 0.9), it may be determined that an abnormal event of double trigger has occurred on the ventilator, and thus abnormality prompt information indicating that double trigger has occurred on the ventilator is sent.

In an embodiment, the ventilator may send the abnormality prompt information, by means of a UI and/or in the form of sound, indicating that double trigger has occurred on the ventilator when determining that the abnormal event of double trigger has occurred, so that the user adjusts ventilation (e.g., the tidal volume, the inspiratory pressure or inspiratory time) to better realize the patient-ventilator synchronization.

In an embodiment, a preset ventilation adjusting strategy may be adopted to adjust the tidal volume/inspiratory pressure/inspiratory time of ventilation and the expiratory trigger sensitivity when the ventilator determines that the abnormal event of double trigger has occurred, for example, the trigger sensitivity is periodically reduced (e.g., in each subsequent respiratory cycle, the trigger sensitivity is reduced to 90% of that in a previous cycle each time) until no double trigger event occurs.

With regard to false trigger, the patient does not make the inspiratory effort, and due to the fact that the trigger sensitivity of the ventilator is set too low, a pipeline leaks or accumulated water of the line shocks, the pressure in the line changes to trigger the ventilator to delivery gas, which is called self-trigger of the ventilator and also called false trigger. Reasons for the false trigger may include, but are not limited to, the following situations:

(1) too lower trigger setting: a pressure change in a ventilator circuit often occurs due to the shake of the line and the shock of accumulated water in the line, causing false trigger, when the trigger sensitivity of the ventilator is set too low;

(2) water accumulation of the line: if the trigger sensitivity of the ventilator is properly set while a large amount of water is accumulated in the line, the pressure in the ventilator circuit may be suddenly reduced to cause self-trigger of the ventilator when the accumulated water is poured into an accumulated water bottle by the shaking the line;

(3) line gas leakage: gas delivery of the ventilator is induced when leakage occurs in each connecting pipeline of the ventilator circuit or gas leakage occurs in an endotracheal intubation gas bag to reduce the pressure in the pipeline to be below the trigger sensitivity;

(4) vibration generated by heart beating: it is generally accompanied when the trigger sensitivity of the ventilator is set too low, and a change in the pressure in the lungs is caused during heart beating so as to trigger the ventilator to delivery gas.

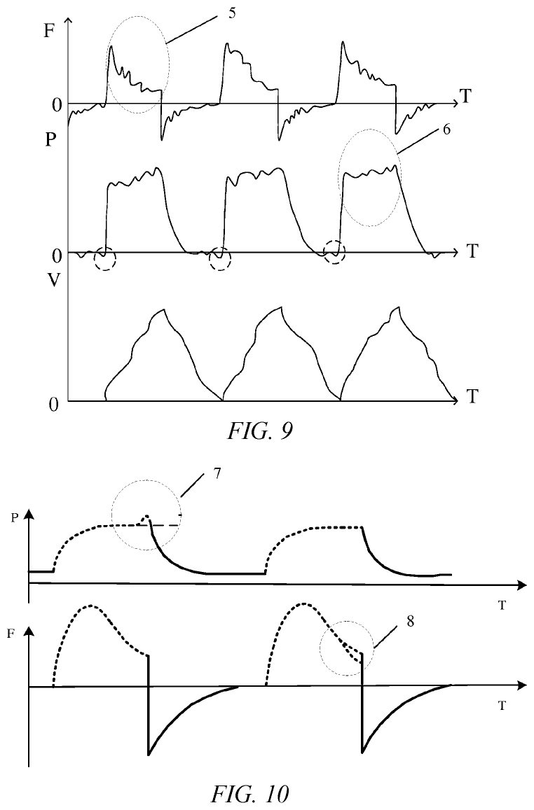

In the case of the false trigger described above, there are similar manifestations in the waveform of the airway pressure over time and the waveform of the airway flow over time. FIG. 9 is a schematic diagram of waveforms of the false trigger provided by an embodiment of the disclosure. Referring to FIG. 9, since the false trigger occurs, changes in the airway pressure and the airway flow may occur repeatedly during gas delivery of the ventilator, as shown by parts indicated by reference numbers 5-6 in FIG. 9.

In an embodiment, when the ventilator detects that at least one of the following situations has occurred by analyzing waveforms of the acquired ventilation parameters (the airway pressure and the airway flow) over time, it may be determined that an abnormal event of false trigger has occurred on the ventilator, that is, it may be determined that the change in the ventilation parameters meets corresponding parameter change conditions:

the airway pressure reaches the pressure trigger sensitivity of the ventilator, and the change in the magnitude of the airway pressure occurs repeatedly during the gas delivery of the ventilator; and

the airway flow reaches the flow trigger sensitivity of the ventilator, and the change in the magnitude of the airway flow occurs repeatedly during the gas delivery of the ventilator.

In an embodiment, a pressure-time waveform graph and a flow-time waveform graph corresponding to the false trigger event may be stored in the ventilator, a corresponding waveform graph may be obtained by monitoring the airway pressure and/or airway flow during the mechanical ventilation by the ventilator, and the obtained waveform graph is matched with the stored corresponding waveform graph of false trigger. For example, the obtained pressure-time waveform graph may be matched with the stored pressure-time waveform graph (within one respiratory cycle) corresponding to the false trigger, and when the similarity obtained by matching reaches a similarity threshold (e.g., 0.9), it may be determined that an abnormal event of inspiratory false trigger has occurred on the ventilator, and thus abnormality prompt information indicating that false trigger has occurred on the ventilator is sent.

In an embodiment, the ventilator may send abnormality prompt information, by means of a UI and/or in the form of sound, indicating that the false trigger has occurred on the ventilator when determining that the abnormal event of false trigger has occurred, so that the user adjusts the trigger sensitivity or repair the line to better realize the patient-ventilator synchronization.

With regard to abnormal inspiration-to-expiration switching, the inspiration-to-expiration switching may be controlled by the medullary respiratory center and may be an involuntary motion, and the abnormal inspiration-to-expiration switching will be caused when the setting of the inspiration-to-expiration switching during the mechanical ventilation does not meet the demand of the patient. The situation that the ventilator detects either early switching or delayed switching may prompt that the flow-based switching percentage of the ventilator is set improperly.

With regard to delayed cycling in the abnormal inspiration-to-expiration switching, the switching flow may be set too low for a patient suffering from tachypnea, so that the inspiratory time is prolonged, the patient may need to do additional exhalation work, and end inspiratory pressure overshoot may be generated in the waveform, or reduction of the airway flow may be suddenly accelerated; FIG. 10 is a schematic diagram of waveforms of delayed cycling provided by an embodiment of the disclosure. Referring to FIG. 10, a part indicated by a reference number 7 in FIG. 10 may refer to that the patient begins to exhale at the end of inhalation, but the ventilator has not switched to the expiratory phase at this time, so that the airway pressure instantaneously increases to generate an end inspiratory pressure overshoot. A part indicated by a reference number 8 in FIG. 10 may refer to that the patient begins to exhale at the end of inhalation, but the ventilator has not switched to the expiratory phase at this time. The exhalation effort may be not sufficient to cause a significant change in pressure, but a sudden drop in the flow, that is, a sudden increase in the change rate of the flow over time, may be obviously detected.

In an embodiment, when the ventilator detects that at least one of the following situations has occurred by analyzing waveforms of the acquired ventilation parameters (the airway pressure and the airway flow) over time, it may be determined that an abnormal event of delayed cycling has occurred on the ventilator, that is, it may be determined that the change in the ventilation parameters meets corresponding parameter change conditions:

the situation of the end inspiratory pressure overshoot occurs in the waveform of the airway pressure over time; and

in the waveform of the airway flow over time, the situation occurs that the change rate of the airway flow exceeds a preset change rate threshold before the inspiration-to-expiration switching.

In an embodiment, the ventilator may determine the occurrence of the delayed cycling when detecting that a rise appears in the airway pressure-waveform or an accelerated drop occurs in a ventilation flow at an inspiration-to-expiration transitional stage.

In an embodiment, a pressure-time waveform graph and a flow-time waveform graph corresponding to the delayed cycling event may be stored in the ventilator, a corresponding waveform graph may be obtained by monitoring the airway pressure and/or airway flow during the mechanical ventilation by the ventilator, and the obtained waveform graph may be matched with the stored corresponding waveform graph of delayed cycling. For example, the obtained pressure-time waveform graph may be matched with the stored pressure-time waveform graph (within one respiratory cycle) corresponding to the delayed cycling, and when the similarity obtained by matching reaches a similarity threshold (e.g., 0.9), it may be determined that an abnormal event of inspiratory delayed cycling has occurred on the ventilator, and thus abnormality prompt information indicating that delayed cycling has occurred on the ventilator is sent.

In an embodiment, the ventilator may send abnormality prompt information, by means of a UI and/or in the form of sound, indicating that the delayed cycling has occurred on the ventilator when determining that the abnormal event of delayed cycling (delayed cycling) has occurred, so that the user adjusts the trigger sensitivity to better realize the patient-ventilator synchronization.

In an embodiment, a preset trigger sensitivity adjusting strategy may be adopted to adjust the trigger sensitivity of the ventilator when the ventilator determines that the abnormal event of delayed cycling has occurred, for example, the trigger sensitivity of the ventilator may be periodically increased (e.g., in each subsequent respiratory cycle, the trigger sensitivity is increased to 110% of that in a previous cycle each time) until no delayed cycling event occurs.

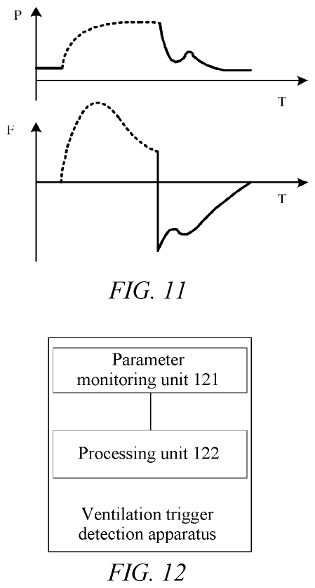

With regard to premature cycling in the abnormal inspiration-to-expiration switching, since the exhalatory trigger sensitivity (a percentage of the peak flow) of the ventilator may be set too high, the ventilator stops delivering the gas and may be switched for expiration when the patient still performs the inhalation action, causing insufficient inspiration for the patient and the patient-ventilator incoordination. It may be manifested by the flow-time waveform that a descending inspiratory branch may decrease to zero in advance and become an expiratory one, while the expiratory phase at an initial stage has the trend of rising again. If the patient's inhalation effort is strong, repeated trigger of the ventilator may be caused. The early switching may lead to the reduction of the tidal volume and polypnea of the patient, shortening the inspiratory time and increasing the respiration work of the patient. FIG. 11 is a schematic diagram of waveforms of premature cycling provided by an embodiment of the disclosure.

In an embodiment, when the ventilator detects that at least one of the following situations has occurred by analyzing waveforms of the acquired ventilation parameters (the airway pressure and the airway flow) over time, it may be determined that an abnormal event of premature cycling has occurred on the ventilator, that is, it may be determined that the change in the ventilation parameters meets corresponding parameter change conditions:

the situation that the airway pressure rises and then drops after the inspiration-to-expiration switching occurs in the waveform of the airway pressure over time; and

in the waveform of the airway flow over time, the situation occurs that the airway flow increases and then decreases after the inspiration-to-expiration switching.

In an embodiment, the ventilator may determine the occurrence of the premature cycling when detecting the situation that a non-monotonic drop appears in the airway pressure-time waveform or a non-monotonic rise appears in the airway flow-time waveform at the inspiration-to-expiration transitional stage.

In an embodiment, a pressure-time waveform graph and a flow-time waveform graph corresponding to the premature cycling event may be stored in the ventilator, a corresponding waveform graph may be obtained by monitoring the airway pressure and/or airway flow during the mechanical ventilation by the ventilator, and the obtained waveform graph may be matched with the stored corresponding waveform graph of premature cycling. For example, the obtained pressure-time waveform graph may be matched with the stored pressure-time waveform graph (within one respiratory cycle) corresponding to the premature cycling, and when the similarity obtained by matching reaches a similarity threshold (e.g., 0.9), it may be determined that an abnormal event of inspiratory premature cycling has occurred on the ventilator, and thus abnormality prompt information indicating that premature cycling has occurred on the ventilator is sent.

In an embodiment, the ventilator may send abnormality prompt information, by means of a UI and/or in the form of sound, indicating that the premature cycling has occurred on the ventilator when determining that the abnormal event of premature cycling has occurred, so that the user adjusts the trigger sensitivity to better realize the patient-ventilator synchronization.

In an embodiment, a preset trigger sensitivity adjusting strategy may be adopted to adjust the trigger sensitivity of the ventilator when the ventilator determines that the abnormal event of premature cycling has occurred, for example, the trigger sensitivity of the ventilator may be periodically reduced (e.g., in each subsequent respiratory cycle, the trigger sensitivity is reduced to 90% of that in a previous cycle each time) until no premature cycling event occurs.

Continuing to describe the ventilation trigger detection apparatus provided by the embodiment of the disclosure, as an example of hardware implementation or software implementation of the ventilator, the ventilation trigger detection apparatus may be provided as a series of modules having a coupling relationship at a signal/information/data level, which will be described below with reference to FIG. 12. Referring to FIG. 12, FIG. 12 is an optional schematic diagram of constitution of the ventilation trigger detection apparatus provided by the embodiment of the disclosure, showing a series of units included in implementing the ventilation trigger detection apparatus, but the structure of the units of the ventilation trigger detection apparatus is not only limited to that shown in FIG. 12, for example, the units therein may be further separated or combined according to different functions to be achieved. Referring to FIG. 12, the ventilation trigger detection apparatus includes:

a parameter monitoring unit 121 may be configured to monitor a ventilation parameter during mechanical ventilation for a user, the ventilation parameter may include at least one of an airway pressure and an airway flow; and

a processing unit 122 may be configured to determine patient-ventilator synchrony during the ventilation according to a change in the ventilation parameter.

In an embodiment, the processing unit may be further configured to analyze a change trend of the obtained ventilation parameter;

and to determine whether patient-ventilator out-of-synchronization occurs during the ventilation according to the change trend of the ventilation parameter.

In an embodiment, the processing unit may be further configured to determine the type of the patient-ventilator out-of-synchronization according to the change trend of the ventilation parameter.

In an embodiment, the type of the patient-ventilator out-of-synchronization includes one or more of ineffective effort, double trigger, false inspiratory trigger, delayed cycling and premature cycling.

In an embodiment, the processing unit may be further configured to determine the occurrence of the ineffective effort when detecting that a valley appears in an airway pressure-time waveform and/or an accelerated rise appears in a ventilation flow-time waveform at an expiratory stage, and inspiratory trigger of a ventilator is not enabled.

In an embodiment, the processing unit may be further configured to determine the occurrence of the double trigger when detecting that two inspiratory pressure waveforms appear in the airway pressure-time waveform at an inspiratory stage and/or a short-time expiratory cycle appears between two inspiratory cycles in the airway flow-time waveform.

In an embodiment, the processing unit may be further configured to determine the occurrence of the delayed cycling when detecting that a rise appears in the airway pressure-time waveform or an accelerated drop occurs in a ventilation flow-time waveform at an inspiration-to-expiration transitional stage.

In an embodiment, the processing unit may be further configured to determine the occurrence of the premature cycling when detecting that a non-monotonic drop appears in the airway pressure-time waveform or a non-monotonic rise appears in the airway flow-time waveform at the inspiration-to-expiration transitional stage.

In an embodiment, the processing unit may be further configured to adjust ventilation trigger setting of the ventilation device or output prompt information about the patient-ventilator out-of-synchronization according to the determined type of the patient-ventilator out-of-synchronization.

An embodiment of the disclosure may further provide a readable storage medium. The storage medium may include: a mobile storage device, a random access memory (RAM), a read-only memory (ROM), a magnetic disk or an optical disk and other media which are capable of storing program codes. The readable storage medium stores executable instructions.

The executable instructions may be configured to implement the ventilation trigger detection method of the embodiment of the disclosure when being executed by the processor.

It should be noted that: the above description relating to the ventilation trigger detection apparatus is similar to the description of the above method and the same as the description of the beneficial effects of the method, which will not be described in detail. Technical details not disclosed in the embodiments of the ventilation trigger detection apparatus according to the disclosure may be referred to the description of the embodiments of the method according to the disclosure.

All or some of the steps of the embodiments may be completed by a program that instructs related hardware. The program may be stored in a computer readable storage medium. When the program is executed, the steps including the above method embodiments are performed. The foregoing storage medium includes: a mobile storage device, a random access memory, a read-only memory, a magnetic disk or an optical disk and other media which are capable of storing program codes.

Alternatively, if implemented in the form of a software function module and sold or used as an independent product, the above integrated unit of the disclosure may also be stored in a computer readable storage medium. Based on such an understanding, the technical solutions in the embodiments of the disclosure essentially, or the part contributing to the related art may be implemented in a form of a software product. The computer software product is stored in a storage medium, including several instructions for instructing a computer device (which may be a personal computer, a server, or a network device) to perform all or some of the methods in the embodiments of the disclosure. The foregoing storage medium includes: a mobile storage device, a RAM, a ROM, a magnetic disk or an optical disk and other media which are capable of storing program codes.

The above descriptions are merely specific embodiments of the disclosure, but the scope of protection of the disclosure is not limited thereto. Changes or substitutions readily figured out by those skilled in the art within the technical scope disclosed in the disclosure shall fall within the scope of protection of the disclosure. Therefore, the scope of protection is set forth by the claims.

1. A ventilation trigger detection method performed by a ventilation device, the method comprising:
monitoring a ventilation parameter during a mechanical ventilation for a user, the ventilation parameter comprising at least one of an airway pressure or an airway flow; and
determining a patient-ventilator synchrony during the mechanical ventilation according to a change in the ventilation parameter.

2. The ventilation trigger detection method of claim 1, wherein determining a patient-ventilator synchrony during the mechanical ventilation according to a change in the ventilation parameter comprises:
analyzing a change trend of the ventilation parameter; and
determining whether a patient-ventilator out-of-synchronization occurs during the mechanical ventilation according to the change trend of the ventilation parameter.

3. The ventilation trigger detection method of claim 2, further comprising:
when the patient-ventilator out-of-synchronization occurs, determining a type of the patient-ventilator out-of-synchronization according to the change trend of the ventilation parameter.

4. The ventilation trigger detection method of claim 3, wherein the type of the patient-ventilator out-of-synchronization comprises one or more of an ineffective effort, a double trigger, a delayed cycling and a premature cycling.

5. The ventilation trigger detection method of claim 4, wherein when the type of the patient-ventilator out-of-synchronization is the ineffective effort, determining a type of the patient-ventilator out-of-synchronization according to the change trend of the ventilation parameter comprising:
determining an occurrence of the ineffective effort when a valley appears in an airway pressure-time waveform or an accelerated rise appears in a ventilation flow-time waveform at an expiratory stage, and an inspiratory trigger of a ventilator is not enabled.

6. The ventilation trigger detection method of claim 4, wherein when the type of the patient-ventilator out-of-synchronization is the double trigger, determining a type of the patient-ventilator out-of-synchronization according to the change trend of the ventilation parameter comprising:
determining an occurrence of the double trigger when two inspiratory pressure waveforms appear in an airway pressure-time waveform at an inspiratory stage or a short-time expiratory cycle appears between two inspiratory cycles in an airway flow-time waveform.

7. The ventilation trigger detection method of claim 4, wherein when the type of the patient-ventilator out-of-synchronization is the delayed cycling, determining a type of the patient-ventilator out-of-synchronization according to the change trend of the ventilation parameter comprising:
determining an occurrence of the delayed cycling when a rise appears in an airway pressure-time waveform or an accelerated drop appears in a ventilation flow-time waveform at an inspiration-to-expiration transitional stage.

8. The ventilation trigger detection method of claim 4, wherein when the type of the patient-ventilator out-of-synchronization is the premature cycling, determining a type of the patient-ventilator out-of-synchronization according to the change trend of the ventilation parameter comprising:
determining an occurrence of the premature cycling when a non-monotonic drop appears in an airway pressure-time waveform or a non-monotonic rise appears in an airway flow-time waveform at an inspiration-to-expiration transitional stage.

9. The ventilation trigger detection method of claim 3, further comprising:
adjusting a ventilation trigger setting of the ventilation device or outputting a prompt information about the patient-ventilator out-of-synchronization according to the determined type of the patient-ventilator out-of-synchronization.

10. The ventilation trigger detection method of claim 9, wherein
an inspiratory trigger sensitivity of the ventilation device is reduced when an ineffective effort occurs, or an inspiratory trigger is enabled when a valley appears in an airway pressure-time waveform or an accelerated rise appears in a ventilation flow-time waveform;
an inspiratory time, an inspiratory pressure or a tidal volume is increased when a double trigger occurs;
an inspiratory trigger sensitivity of the ventilation device is increased when a delayed cycling occurs; and
an inspiratory trigger sensitivity of the ventilation device is reduced when a premature cycling occurs.

11. A ventilation trigger detection apparatus, comprising:
a parameter monitoring unit configured to monitor a ventilation parameter during a mechanical ventilation for a user, the ventilation parameter comprising at least one of an airway pressure or an airway flow; and;
a processing unit configured to determine a patient-ventilator synchrony during the ventilation according to a change in the ventilation parameter.

12. The ventilation trigger detection apparatus of claim 11, wherein
the processing unit is further configured to:
analyze a change trend of the ventilation parameter; and
determine whether a patient-ventilator out-of-synchronization occurs during the mechanical ventilation according to the change trend of the ventilation parameter.

13. The ventilation trigger detection apparatus of claim 12, wherein
the processing unit is further configured to determine a type of the patient-ventilator out-of-synchronization according to the change trend of the ventilation parameter.

14. The ventilation trigger detection apparatus of claim 13, wherein the type of the patient-ventilator out-of-synchronization comprises one or more of an ineffective effort, a double trigger, a false trigger, a delayed cycling and a premature cycling.

15. The ventilation trigger detection apparatus of claim 14, wherein
the processing unit is further configured to determine an occurrence of the ineffective effort when detecting that a valley appears in an airway pressure-time waveform or an accelerated rise appears in a ventilation flow-time waveform at an expiratory stage, and an inspiratory trigger of a ventilator is not enabled.

16. The ventilation trigger detection apparatus of claim 14, wherein
the processing unit is further configured to determine an occurrence of the double trigger when detecting that two inspiratory pressure waveforms appear in the airway pressure-time waveform at an inspiratory stage or a short-time expiratory cycle appears between two inspiratory cycles in the airway flow-time waveform.

17. The ventilation trigger detection apparatus of claim 14, wherein
the processing unit is further configured to determine an occurrence of the delayed cycling when detecting that a rise appears in the airway pressure-waveform or an accelerated drop occurs in a ventilation flow-time waveform at an inspiration-to-expiration transitional stage.

18. The ventilation trigger detection apparatus of claim 14, wherein
the processing unit is further configured to determine an occurrence of the premature cycling when detecting that a non-monotonic drop appears in the airway pressure-time waveform or a non-monotonic rise appears in the airway flow-time waveform at the inspiration-to-expiration transitional stage.

19. The ventilation trigger detection apparatus of claim 15, wherein
the processing unit is further configured to adjust a ventilation trigger setting of the ventilation device or output a prompt information about the patient-ventilator out-of-synchronization according to the determined type of the patient-ventilator out-of-synchronization.

20. A ventilation device, comprising a ventilation trigger detection apparatus, a gas source, an inspiratory branch, an expiratory branch and a respiration line, wherein
the gas source supplies a gas during a mechanical ventilation;
the inspiratory branch is connected to the gas source to provide an inspiration path during the mechanical ventilation;
the expiratory branch provides an expiration path during the mechanical ventilation;
the respiration line is connected to the inspiratory branch and the expiratory branch respectively, and used for delivering the gas to a user or exhausting the gas from a user during the mechanical ventilation; and
the ventilation trigger detection apparatus is connected to the inspiratory branch and the expiratory branch respectively, and comprises:
a parameter monitoring unit configured to monitor a ventilation parameter during the mechanical ventilation for a user, the ventilation parameter comprising at least one of an airway pressure or an airway flow; and
a processing unit configured to determine a patient-ventilator synchrony during the mechanical ventilation according to a change in the ventilation parameter.
